(19) United States
(12) Patent Application Publication
(10) Pub. No.: US 2022/0273847 A1
(43) Pub. Date: Sep. 1, 2022

(54) MATERIAL FOR A BONE IMPLANT

(71) Applicant: stimOS GmbH, Radolfzell (DE)

(72) Inventors: Dietmar Schaffarczyk, Radolfzell (DE); Jennifer Knaus, Radolfzell (DE)

(21) Appl. No.: 17/628,323

(22) PCT Filed: Jul. 17, 2020
(86) PCT No.: PCT/DE2020/100629

§ 371 Date: Jan. 19, 2022

(30) Foreign Application Priority Data
Jul. 19, 2019 (DE) 10 2019 119 607.8

(51) Int. Cl. A61L 27/46; A61L 27/06; A61L 27/34

(52) U.S. Cl. CPC A61L 27/46; A61L 27/06; A61L 27/34; A61L 2430/02; A61L 2420/04

(57) ABSTRACT

A material for a bone implant includes a surface which contains a metal-based material, a metal alloy, an oxide ceramics material, a polymer material, a composite material or combinations thereof. An organic polymer matrix is covalently bonded to the surface. A substance is linked with the organic polymer matrix for binding embedded metal ions or nanoparticles. A calcium phosphate is embedded in the organic polymer matrix. As a result, the material for the bone implant is biocompatible and corrosion can be slowed down or even prevented.

The invention relates to a material for a bone implant, to a method for producing such a material, to a bone implant comprising such a material, and to the use of such a material.

The use of implant materials in people is increasing continuously. This trend is emphasizing the need to look for high-quality bone substitute materials which are stable and functional. The requirements imposed on high-quality, functionally appropriate bone implants are diverse, and it is difficult to meet all of the requirements with one material. At the same time, the functionality of an implant material is difficult to predict, since the natural process of bone wound healing and implant incorporation is highly complex and not completely well enough understood.

There are numerous factors determining the success or failure of a bone implant, and the relationships between these factors are complex and again not yet entirely understood. They include the material properties (chemical, mechanical, and tribological properties), the biocompatibility, the immunogenicity and osseointegration of the implant materials, the health condition of the patient, and the competence of the surgeon. In order for high biocompatibility to be achieved, the material and/or its breakdown products should not be toxic, carcinogenic or teratogenic. Inflammatory responses, immunogenic responses or other negative or adverse responses should not be triggered either in the implant environment or the rest of the body. If individual particles detach from the implant, they too should not trigger any of the aforementioned responses, and should also either be amenable to breakdown in the body or at least secretable, in order to avoid permanent accumulation and attachment/incorporation in the body or an aseptic loosening of the endoprosthesis.

In spite of their excellent clinical performance, doubts have arisen about metal-based implant materials in terms of their long-term compatibility within the tissue/bone and their potential local and systemic side effects. Titanium particles in the tissue have been associated, for example, with monocyte and macrophage activation and with the accompanying release of mediators of bone resorption or hypersensitivity responses. Such metal particles may be released because of erosion, contamination, abrasion or damage to the metal-based bone implant materials during their service life or during the implantation process. In this context, the corrosion of metallic implant materials has to date posed a challenge which, despite attempts to meet it with diverse methods, has nevertheless not yet been resolved.

Corrosion in this context describes a process describing the gradual decomposition of the material as a result of electrochemical attacks or abrasion within the body of the patient. The variations in the local pH owing to diverse reasons have been identified as a source of corrosion events. Such variations may be brought about, for example, by gradual imbalances in the physiochemical composition of the local body fluid (e.g., fraction of dissolved gases such as oxygen) or general imbalances of the biological system as a result, for example, of disease or bacterial infections.

The direct corrosion of material here may be accelerated by concomitant processes, such as abrasion or wear, for example, and so results in what is called tribocorrosion. This may take place, for example, through repeated cyclic loading (by walking, for example), which damages the oxide layer naturally protecting the metal or wears it away entirely, so exposing the reactive, unpassivated metal. This layer is reestablished by the initially oxygen-rich body fluid. As a result of this, however, the local oxygen concentration is reduced and natural passivation becomes more difficult. Accordingly, there may be a local acidification in pH, which in turn accelerates the corrosion process. In this case, over time, the material continues to be attacked. Furthermore, metal particles may be abraded more easily as a result of this, and may diffuse away from the surface and be a trigger, in some cases, of inflammatory responses.

As well as the development of new implant materials having specific properties intended to slow down or even prevent such corrosion effects, there is a focus in research on the development of surface coatings.

On this basis, it is an object of the present invention to provide a material for a bone implant that firstly comprises biocompatible components bonded covalently to the surface. Secondly, any possible corrosion is to be slowed down or even prevented. It is a further object of the present invention, moreover, to provide a corresponding production method allowing such a material to be produced simply and with high yield. Additionally, a further object of the present invention is the provision of a bone implant with such a material, which is highly compatible and long-lived. A further object of the present invention is the diverse and simple use of such a material.

These objects are achieved in accordance with the invention with a material for a bone implant, with a production method for such a material, with an implant with such a material, and by the use of such a material, with the features of the independent claims. Advantageous embodiments and benefits of the invention are apparent from the further corresponding dependent claims, from the drawing, and from the description.

The invention starts from a material for a bone implant, comprising: (a) a surface comprising a material selected from the group consisting of metal-based materials, metal alloys, oxide ceramic materials, polymer materials, composite materials, or combinations thereof, (b) an organic-polymeric matrix bonded covalently to this surface, (c) a substance incorporated or attached to this organic-polymeric matrix and binding metal ions or nanoparticles, and (d) calcium phosphate incorporated into this organic-polymeric matrix.

By means of the material of the invention it is possible to provide a material which has high compatibility by virtue of its biocompatibility. It additionally has self-regenerating and antibacterial properties. Moreover, it is able to alleviate, or even entirely prevent, negative effects of corrosion. The specific composition of the material of the invention allows it to be tailored for/to particular areas of use.

The protective effect of the material of the invention is based, accordingly, on multiple barrier functions. The covalent attachment of the organic network to the surface, and also the extensive cohesion of the network, prevents the coating detaching from the surface and also the breakdown and diffusion of material, such as metallic components, such as metal ions or metal nanoparticles. If nanoparticles of the bulk material should then diffuse away from the surface as a result of corrosion or abrasion, the polymer matrix is able accordingly to prevent far-reaching diffusion of the particles into the surrounding tissue. Furthermore, the gel layer possesses the property of independently closing fissures. This leads to a continuous gel layer again if the surface is damaged.

A further protection is represented by the calcium phosphate. Should the pH be reduced on the surface of the implant, the calcium phosphate layer can also dissolve. If metal ions should then diffuse from the surface through the network of the organic layer, they are able to form, with the dissolved phosphate ions, insoluble metal phosphates and so to prevent the far-reaching diffusion of the metal ions into the surrounding tissue. A mechanism of this kind has been proposed for adsorbed calcium phosphate coatings on metal surfaces. In these cases, however, the proposed mechanism may result in the detachment and hence the failure of the coating, owing to the noncovalent nature of the coating. When the material according to the invention is used, such total failure of the coating is prevented because of the covalent nature of the organic layer and because of the partial composite nature of the mineral layer.

The use of certain terms in the singular or plural in the claims or the description is not intended to restrict the scope of protection of the patent or patent application only to this specifically stated number. The scope of protection of the invention is also intended to relate to a singular, multiple or any other number of the structure in question.

The terms “material for a bone implant” and “bone implant material” are used synonymously here.

The material of the invention is applied to solid, usually metal-based materials or bodies (main structure) which are used as a bone implant. The surface from subsection (a) here may be a surface of this body, or a surface of a layer applied on this body. These bodies may have any desired or necessary three-dimensional form.

The total surface of the material of the invention for bone implants preferably comprises or consists of the material defined in subsection (a) above. Suitable materials to which the material of the invention or else, where appropriate, only the organic-polymeric matrix may be applied may be all of the metal-based materials, metal alloys, (oxide) ceramic materials, polymer materials, composite materials, or combinations thereof that to the skilled person are known or considered to be usable.

Examples thereof include, as metals: titanium/stainless steel; as ceramics: zirconia (zirconium dioxide); as polymer: polyetherketone (PEK) and the entire PEK family, but especially: polyetheretherketone (PEEK), polyetherketone-ketone (PEKK), polyetherketone-etherketoneketone (PEKEKK), carbon fiber reinforced PEEK (CFR-PEEK), PEEK composites, glass fiber reinforced polymers, polyethylene (PE), ultra-high-molecular weight polyethylene (UHMWPE), polyorthoesters, polymethyl methacrylate (PMMA), polyethylene terephthalate (PET), or polyamides (PA). In one preferred embodiment the material is titanium or a composite material thereof. This/these material/s is/are the current gold standard in clinical application, as it/they has/have good biocompatible properties and is/are therefore highly suitable as bone implant material.

An organic-polymeric matrix refers here to a network of molecules which is made up to a major part (more than 50%) of at least one main building block with a carbon framework, this framework linking and/or crosslinking a main building block or two or more main building blocks multiply and/or occurring in succession in a chain. This may be a substance or a substance mixture which occurs naturally, or a synthetically produced substance/substance mixture. The organic-polymeric matrix preferably covers the entire surface of the material from section (a), hence allowing the material to be protected from the physiological conditions of the implantation site.

The organic-polymeric matrix may be any matrix considered by the skilled person to be usable, or may comprise any materials considered to be usable, such as collagens, polysaccharides or polycatechols, for example.

The organic-polymeric matrix bonded covalently to the surface advantageously comprises collagen, preferably type I collagen, and/or gelatin. Collagen is the organic component of natural bone, which consists of said collagen to an extent of around 95%. The remaining components of natural bone, to an extent of around 5%, are proteoglycans and other adhesion-promoting glycoproteins. Gelatin is a denatured form of collagen, and in comparison to the latter is more favorably priced and easier to handle. In addition, however, gelatin still possesses a number of advantageous properties of natural collagen, such as, for example, the formation of protein fibers in solution similar to those of the natural collagen. Moreover, gelatin is able to form hydrogels, which under specific circumstances exhibit self-repair properties. The term “self-repair” or “self-repairing” as used herein denotes the property, on the part of the material for a bone implant, of independently closing “injuries” such as fissures, for example, within the matrix (gel layer). In this way a continuous gel layer is reestablished. The use of gelatin as a matrix material is therefore preferred. Through the use of such gel-forming materials, the material of the invention for bone implants likewise has self-repair properties.

The gelatin and the collagen may be chemically modified. By way of free chemical groups in the amino acids, such as amine, acid or hydroxyl groups, for example, it is possible to introduce further functionalizations into the coating via established coupling chemistry by way of 1-ethyl-3-(3-dimethylaminopropyl)carbodiimide (EDC), or similar, analogous methods. This further layer of modified protein enables, on the one hand, an increase in the fraction of ion-binding substances additionally to those which are located in the polysaccharide layer (see below). However, it also enables the introduction of further combinatorial functionalizations, such as the introduction of cell growth promoter substances or antimicrobial substances, for example.

According to one preferred embodiment of the invention, the organic-polymeric matrix bonded covalently to the surface comprises a polysaccharide and/or a modified polysaccharide. A further component of the organic-polymeric matrix of the material of the invention may therefore be a polysaccharide. As a result, a class of substance having a multiplicity of positive properties may be employed. As well as antibacterial properties, some polysaccharides are likewise ascribed nonallergenic, nontoxic, wound-healing, hemostatic, bacteriostatic, and fungicidal material properties. These make them suitable as biomaterials for use for wound management, as hemostatic materials or as scaffold structures for artificial tissue generation.

The polysaccharide of the material of the invention might, for example, be chitosan, alginic acid, alginate, hyaluronic acid, hyaluronate, pectin, carrageenan, agarose, and amylose. Also possible would be any other glycosaminoglycan, such as heparin/heparan sulfate, chondroitin sulfate/dermatan sulfate or keratan sulfate. Also conceivable are hemicelluloses, such as xylanes or mannans after a carboxy-functionalization, or else xanthan, gellan, fucogalactan, or welan gum. Moreover, all conceivable mixtures may also be employed.

In one preferred embodiment the polysaccharide is selected from a group consisting of chitosan, alginic acid, alginate, hyaluronic acid, and hyaluronate. This allows many different compounds to be employed, which can be selected individually through their specific properties.

It may be advantageous, moreover, for the polysaccharide to be a chemically modified polysaccharide. In this context, “chemically modified” is intended to mean that the polysaccharide had undergone synthetic, laboratory-chemical alteration of a sugar of the polysaccharide, such as, for example, on a free group, e.g., hydroxyl, aldehyde or acid group. In this way it is possible to extend the spectrum of use. For example, inactive groups can be modified in a targeted way to become active groups, or an unwanted property can be eliminated. Moreover, further functionalizations can be introduced into the material, and the degree of crosslinking within the matrix can be controlled. Such functionalizations may entail, for example, the introduction of ion-binding substances such as pyrocatechols.

In one preferred realization of the invention, the organic-polymeric matrix bonded covalently to the surface comprises a polycatechol. The term “polycatechol” is also intended to comprehend polycatecholamine. Similarly to the properties of the polysaccharides, polycatechols and polycatecholamines are ascribed antibacterial properties. On account of their high binding capacity to numerous different material surfaces, polycatechols may be used as a coating for the introduction of functionalizations. These include, for example, use as antifouling coatings.

The additional modification of the matrix with catechols and/or catecholamines likewise slows down or prevents the onward diffusion of metal ions and nanoparticles from the surface. Catechols naturally possess the property of strong binding of metal ions and metal nanoparticles. This binding is intensified under slightly acidic ambient conditions. If the ambient pH should lower and, consequently, if metal ions or metal nanoparticles should dissolve and diffuse away from the direct surface, they are captured to an increased extent within the matrix/gel layer.

The polycatechol preferably has a pyrocatechol main structure, the pyrocatechol being selected from a group consisting of dopamine, norepinephrine or L-3,4-dihydroxyphenylalanine (L-DOPA). Accordingly such polycatechols or polycatecholamines may be prepared, for example, by simple oxidation of pyrocatechols, such as dopamine, norepinephrine or L-3,4-dihydroxyphenylalanine (L-DOPA), for example. In this case an increased oxygen content in the solution used may be enough of itself to initiate a polymerization. Also conceivable, however, are oxidizing agents such as ammonium peroxydisulfate or sodium periodate, for example.

The matrix may thus comprise either collagen/gelatin or a polysaccharide or a polycatechol, or a combination of two substances or three substances in each case from one of these classes of substance. Layer construction or a mixture would be possible. The mixture of the attached matrix substances can in this case be varied steplessly and hence the profiles of properties can be adapted as well.

In one preferred realization of the invention, the organic-polymeric matrix bonded covalently to the surface comprises collagen, preferably type I collagen, and/or gelatin, a polysaccharide or a modified polysaccharide, and a polycatechol. As a result of this there are numerous possibilities for combination in the composition of the matrix, hence allowing the material and/or the function of the implant to be adapted or tailored precisely to the requirement at the implantation site. Gelatin, for example, has self-healing activity, chitosan and polydopamine antibacterial activity, and the polydopamine also acts as a contact mediator between the gelatin and the chitosan. Additionally, the polydopamine may also serve as a metal ion scavenger.

The polysaccharides and polycatechols and/or polycatecholamines used may act antibacterially here and so counteract a reduction in the pH at the implantation site owing to bacteria. It is possible accordingly to slow down or even prevent the effect of further corrosion.

In one preferred embodiment of the invention, the substance which binds metal ions or nanoparticles is a pyrocatechol. Accordingly a very strongly binding substance can be employed. The pyrocatechol may be any pyrocatechol considered by the skilled person to be usable; preferably the pyrocatechol is selected from a group consisting of protocatechuic alcohol, protocatechualdehyde, protocatoic acid, 3-(3,4-dihydroxyphenyl)propionic acid, and 3,4-dihydroxyphenylacetic acid. These substances bind strongly to metals. The binding may be reinforced by the presence and/or establishment of slightly acidic ambient conditions. The pyrocatechol may be introduced, for example, as a modification of the collagen and/or of the polysaccharide on, for example, a free group, e.g., hydroxyl, aldehyde or acid groups.

The use of the substance which binds metal ions or nanoparticles may even make it possible for the organic-polymeric matrix not to require any polycatechol, since the capacity of the latter to bind metal ions or metal nanoparticles is taken over by the substance which binds metal ions or nanoparticles. Another consequence of this is that the polycatechol in the matrix (subsection (b)) may take over the function of the substance which binds metal ions or nanoparticles (subsection (c)) and there may be no need to add a further substance as per subsection (c).

It may be advantageous, furthermore, for the organic-polymeric matrix to be bonded to the surface via a linker. This produces a stable connection between the surface, usually that of the main implant body, and the organic-polymeric matrix. Any linker considered by the skilled person to be usable may be used here. The organic-polymeric matrix may be coupled to the materials defined in subsection (a) by way of linker molecules mounted on the surface, such as, for example, pyrocatechol, phosphonic acid, phosphoric acids, or organosilane molecules. These may be mounted on the surface by incubation of the surface in a solution of the corresponding linker. In this way, chemical groups with selective functionality may be introduced on the surface. It is possible, for instance, for the polysaccharide to be attached to the implant via an ester bond or amide bond, using the hydroxyl group in 6-position that is present in the majority of polysaccharides. In this case, for example, treatment may take place with EDC, hexamethylenediamine (HMDA) or adipic dihydrazide (ADH). HMDA and ADH are both diamide linkers. Since ADH has a lower basicity than HMDA, coupling is even possible in the acidic pH range of 4.8. The two linkers therefore address a coupling chemistry in different pH ranges. A different linker may therefore be needed for the attachment of the polysaccharide, according to requirements.

In this way the entire repertoire of mixtures of the individual components of the matrix of the material of the invention is available for covalent bonding to the implant.

The material of the invention for a bone implant further advantageously comprises calcium phosphate incorporated into the stated organic-polymeric matrix. In this context it is possible for all mineral forms of calcium orthophosphate to be employed. The calcium phosphate is preferably selected from the group consisting of amorphous calcium orthophosphate (ACP), dicalcium phosphate dihydrate (DCPD; brushite), octacalcium phosphate, and hydroxylapatite, including with partial fluoride, chloride, strontium or carbonate substitution. Particularly preferred are amorphous calcium phosphate (ACP), hydroxylapatite, and octacalcium phosphate. Methods for incorporating the stated calcium phosphates into a corresponding matrix are described below.

As a result of the direct mineralization of the calcium phosphate within the matrix, it is anchored directly on the surface. This avoids common problems with simple coating methods known from the prior art. Those methods frequently feature not only low adhesion of the calcium phosphates on the implant material but also a limited cohesion within the individual layers of calcium phosphate. This greatly increases the risk of delamination. Furthermore, there is an increased risk, with such simple coatings, that fissures may form rapidly as a result of loading, to the detriment of the corrosion protection of the surface.

A good and functional coating of the surface with the organic-polymeric matrix may be achieved advantageously if the organic-polymeric matrix has a layer thickness of between 0.5 micrometers (μm) and 50 μm, preferably between 1 μm and 20 μm, and more preferably of 10 μm.

A highly promising combination for the structure of the material of the invention would be, for example, a layer structure composed of polydopamine, pyrocatechol-modified chitosan and pyrocatechol-modified gelatin, which can be crosslinked by addition of crosslinking substances. For this purpose, for example, treatment may take place with EDC, HMDA, ADH, formaldehyde or glutaraldehyde. Via this crosslinking it is then also possible subsequently for further layers to be attached, by simple impregnation of the layer with the crosslinker, washing, and application of the next layer for coupling. In between, steplessly, all combinations of polysaccharides, modified polysaccharides, gelatins, modified gelatins, and polydopamine are available by way of a common chemical attachment via ester bonds.

According to one advantageous aspect of the invention, the material for a bone implant comprises: (a) a surface of titanium, (b) an organic-polymeric matrix bonded covalently to this surface and comprising pyrocatechol-modified gelatin, pyrocatechol-modified chitosan, and polydopamine, (c) pyrocatechol molecules as the substance which is incorporated or attached to the organic-polymeric matrix and binds metal ions or nanoparticles, and (d) hydroxylapatite incorporated into this organic-polymeric matrix. This combination of materials advantageously combines a robust surface material with a self-healing, antibacterial, bonelike matrix which scavenges metal ions and nanoparticles.

The invention also starts from a method for producing an above-described material for a bone implant. This method comprises at least the steps of: (a) providing a surface comprising a material selected from the group consisting of metal-based materials, metal alloys, oxide ceramic materials, polymer materials, composite materials, or combinations thereof, (b) covalently coupling an organic-polymeric matrix to this surface, (c) introducing and/or coupling a substance which binds metal ions or nanoparticles into/to the organic-polymeric matrix, and (d) mineralizing the organic-polymeric matrix with calcium phosphate.

The material can be produced simply and efficiently by means of the method of the invention. This material, owing to its biocompatibility, is highly compatible. It also has self-regenerating and antibacterial properties. Furthermore, it is able to alleviate or even entirely prevent negative effects of corrosion. The specific composition of the material of the invention allows it to be tailored for specific areas of use.

Where the organic-polymeric matrix comprises a polycatechol, moreover, the polycatechol is prepared in step (b) by means of simple oxidation of at least one pyrocatechol. An increased oxygen content in the solution used may be sufficient for this purpose in order to initiate a polymerization. The polycatechol can therefore be simply prepared.

The substance which binds metal ions or nanoparticles may be introduced and/or coupled into/to the organic-polymeric matrix in accordance with step (c) by incubation of the organic-polymeric matrix in the corresponding substance solution, with subsequent incubation in a solution of a coupling mediator. The coupling mediator may be EDC, HMDA, ADH, formaldehyde or glutaraldehyde, for example.

The invention also starts from a bone implant comprising a solid material and/or a solid body on which the bone implant material of the invention is applied. This allows a bone implant to be provided which is compatible and particularly long-lived.

The invention, moreover, starts from the use of the material of the invention as a bone implant material, hence allowing the provision of a material for an area in which high compatibility and longevity are important.

The properties, features and advantages of this invention that are described above, and also the manner in which they are achieved, become comprehensible more distinctly and with greater clarity in association with the description hereinafter of the exemplary embodiments, which are elucidated in more detail in association with the drawings. The examples given in association in the description below are not intended to restrict the invention to the combination of features specified therein, including not in relation to functional features. The drawings, the description, and the claims contain numerous features in combination. The skilled person will expediently also consider the features individually and group them together to form rational further combinations.

### IN THE DRAWINGS

FIG. 1 shows a schematic representation of a construction of a material for a bone implant with an organic-polymeric matrix, with a layer-by-layer construction of the individual substances,

FIG. 2 shows a schematic representation of the construction of the material for a bone implant from FIG. 1, in mineralized form,

FIG. 3 shows a schematic representation of an alternative construction of a material for a bone implant with an organic-polymeric matrix, as a mixture of the individual substances, and

FIG. 4 shows a schematic representation of the construction of the material for a bone implant from FIG. 3, in mineralized form.

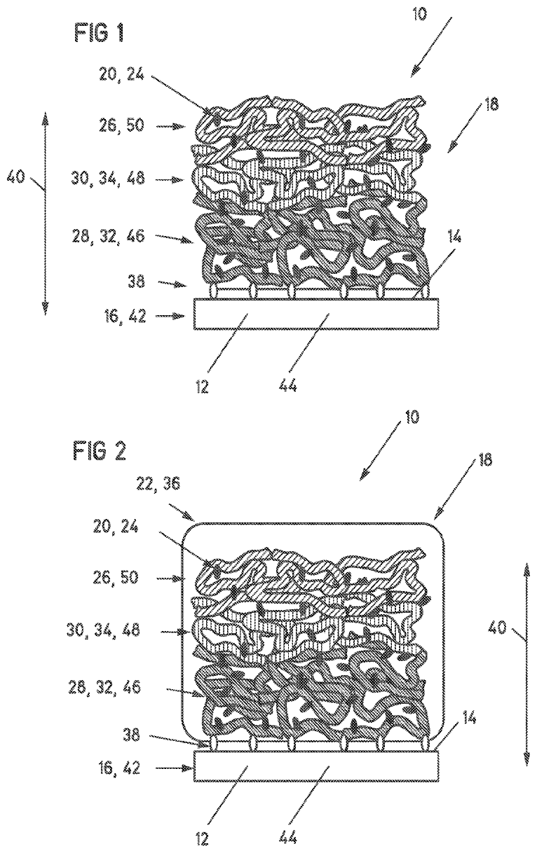

FIG. 1 shows, in a schematic representation, a construction of a material 10 for a bone implant 12 (not shown in detail) with an organic-polymeric matrix 18, with a layer-by-layer construction of individual substances, such as gelatin 26, chitosan 32, and polydopamine 34.

The material 10 for the bone implant 12, which is formed, for example, of a solid material or of a three-dimensional body 44 (not shown in detail), comprises a surface 14, comprising a material 16 selected from the group consisting of metal-based materials, metal alloys, oxide ceramic materials, polymer materials, composite materials, or combinations thereof, and here specifically titanium 42.

Applied to the surface 14, as a surface coating, is an organic-polymer matrix 18 bonded covalently to this surface 14, application taking place layer by layer or in at least three layers 46, 48, 50. The organic-polymeric matrix 18 comprises collagen and/or gelatin 26 (layer 50), a polysaccharide 28 or a modified polysaccharide 28 (layer 46), and a polycatechol 30 (layer 48). Moreover, the organic-polymeric matrix 18 covers the entire surface 14 of the material 16.

In this case the polysaccharide 28 is selected from a group consisting of chitosan 32, alginate, hyaluronic acid, alginic acid, hyaluronate, pectin, carrageenan, agarose, amylose, heparin/heparan sulfate, chondroitic sulfate/dermatan sulfate, keratan sulfate, xylans or mannans after a carboxy-functionalization, xanthan, gellan, fucogalactan, or welan gum, and here by way of example is chitosan 32.

The polycatechol 30 has a pyrocatechol main structure, the pyrocatechol 24 being selected from a group consisting of dopamine 34, norepinephrine or L-3,4-dihydroxyphenylalanine (L-DOPA). In this exemplary embodiment, shown by way of example, the pyrocatechol main structure is based on dopamine 34, and so the polycatechol 30 is polydopamine 34.

The organic-polymeric matrix 18 additionally has a layer 50 of gelatin 26. The polycatechol 30 or polydopamine 34 serves here as a connector between the layer 46 of chitosan 32 and the layer 50 of gelatin 26. In this exemplary embodiment, the chitosan 32 is first applied to the surface 14, then the contact mediator polydopamine 34, and subsequently the gelatin 26. In principle, however, it is also possible for the gelatin 26 to be applied first, and the chitosan 32 after the polydopamine 34.

In order to capture any detaching metal which diffuses away, the organic-polymeric matrix 18 comprises a substance 20 which is incorporated or attached to the organic-polymeric matrix 18 and which is able to bind metal ions or nanoparticles. This substance 20 binding the metal ions or nanoparticles is a pyrocatechol 24, preferably selected from a group consisting of protocatechuic alcohol, protocatechualdehyde, protocatechuic acid, 3-(3,4-dihydroxyphenyl)propionic acid, and 3,4-dihydroxyphenylacetic acid. The polycatechol 30 or polydopamine 34 of the layer 48 is also able to bind metal ions or metal nanoparticles. Pyrocatechol molecules 24 preferably serve as the substance 20 which is incorporated or attached to organic-polymeric matrix 18 and binds metal ions or nanoparticles.

The substance 20 may act as a crosslinker of the collagen or of the gelatin 26, of the polycatechol 30 or polydopamine 34, and of the polysaccharide 28 or the chitosan 34, and therefore represents modifications of these molecules.

In the exemplary embodiment shown by example here, the organic-polymeric matrix 18 bonded covalently to the surface 14 of titanium 42 therefore comprises a layer 50 of pyrocatechol-modified gelatin 26, a layer 48 of pyrocatechol-modified polydopamine 34, and a layer 46 of pyrocatechol-modified chitosan 32.

The organic-polymeric matrix 18 is bonded to the surface 14 via a linker 38, which is selected from the group consisting of pyrocatechol, phosphonic acid, phosphoric acid, and organosilane molecules. It is preferably a silane linker 38.

As can be seen in FIG. 2, which is a schematic representation of the construction of the material 10 for the bone implant 12, in mineralized form, the organic-polymeric matrix 18 comprises incorporated calcium phosphate 22. The calcium phosphate 22 is selected from the group consisting of calcium orthophosphate 22 in all mineral forms or from the group consisting of amorphous calcium orthophosphate (ACP), dicalcium phosphate dihydrate (DCPD; brushite), octacalcium phosphate, and hydroxylapatite 36, including with partial fluoride, chloride, strontium or carbonate substitution, and combinations thereof. According to the embodiment shown, the calcium orthophosphate 22 is hydroxylapatite 36.

The organic-polymeric matrix 18 has a layer thickness 40 of between 0.5 micrometers (μm) and 50 μm, preferably between 1 μm and 20 μm, and very preferably of 10 μm.

A method of the invention for producing the material 10 for a bone implant 12, comprises at least the steps of:

(a) providing a surface 14 comprising a material 16 selected from the group consisting of metal-based materials, metal alloys, oxide ceramic materials, polymer materials, composite materials, or combinations thereof,

(b) covalently coupling an organic-polymeric matrix 18 to this surface 14,

(c) introducing and/or coupling a substance 20 which binds metal ions or nanoparticles into/to the organic-polymeric matrix 18, and

(d) mineralizing the organic-polymeric matrix 18 with calcium phosphate 22.

Where the organic-polymeric matrix 18 comprises a polycatechol 30, the polycatechol 30 is prepared in step (b) by means of simple oxidation of at least one pyrocatechol 24.

The substance 20 which binds metal ions or nanoparticles may be introduced and/or coupled into/to the organic-polymeric matrix 18 by incubation of the organic-polymeric matrix 18 in the corresponding substance solution, with subsequent incubation in a solution of a coupling mediator. The coupling mediator may be EDC, HMDA, ADH, formaldehyde or glutaraldehyde, for example.

Described below by way of example is the production of the material 10:

Coating of the titanium-based surface 14 with silane linkers 38:

The surface 14/substrate used was a substrate coated by vapor deposition with 200 nanometers (nm) of titanium 42, and also metal flakes of titanium 42. The reaction is carried out as represented in scheme 1. In this case, first of all, the (3-aminopropyl)trimethoxysilane (APTS) is hydrolyzed in a slightly acidic medium at a pH of 4 for 15 minutes (min) at room temperature.

Simultaneously, in parallel, the titanium substrates are cleaned with ethanol and water and then incubated in 2 mol (M) NaOH to activate the surface. The cleaned and dried titanium substrates are then immersed into silane solution and incubated at room temperature for 1 hour (h). The unbound silane linker molecules are washed off subsequently with water.

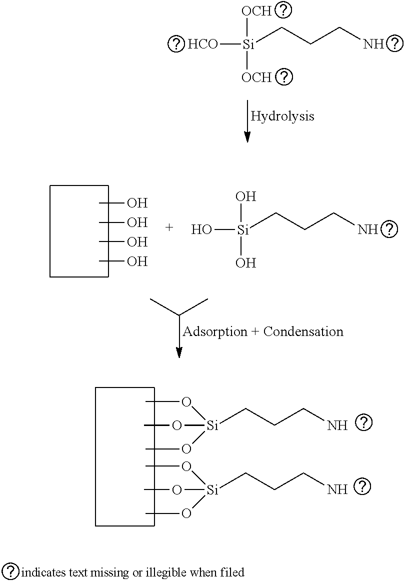

Scheme 1: schematic representations of the reaction pathway of the coating of the titanium-based substrates with silane linkers

Coupling of a layer 46 of chitosan 32:

The coupling of chitosan 32 (or alternatively modified chitosan 32) is accomplished using 1-ethyl-3-(3-dimethylaminopropyl)carbodiimide hydrochloride (EDC). This is a widespread and commercially available coupling reagent which is frequently employed for the chemical coupling of, for example, proteins and peptides, oligonucleotides. Together with N-hydroxysuccinimide (NHS), specifically, a reaction of carboxylates and amines is promoted, to form an amide bond. EDC coupling reactions are carried out typically under acidic reaction conditions (pH 4.5 to 5.5). The reaction scheme for the coupling of chitosan 32 to the silane surface is shown in scheme 2.

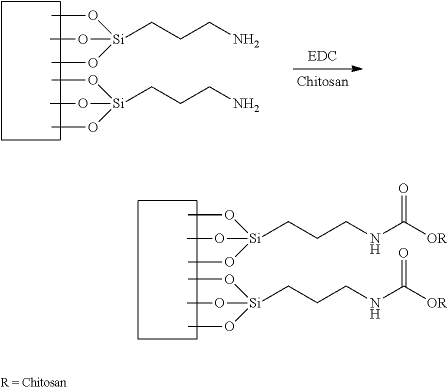

Scheme 2 schematic representations of the reaction pathway of the coupling of chitosan 32 to a silane-modified titanium surface 14

Coupling of a layer 48 of polydopamine 34:

The attachment of a layer 48 of polydopamine 34 is accomplished by incubation of the titanium-silane linker-chitosan material in a solution of L-DOPA, which is subsequently polymerized by addition of an oxidizing agent. The surface is subsequently washed thoroughly with water.

Coupling of a layer 50 of gelatin 26:

The further attachment of a layer 50 of gelatin 26 (or of modified gelatin 26) is accomplished by incubation of the above-produced material in a solution of gelatin 26 at 40° C. Immediately thereafter a crosslinker is added, such as EDC or hexamethylene diisocyanate, for example. The surface is subsequently washed thoroughly with water.

Modification of chitosan 34 or gelatin 26:

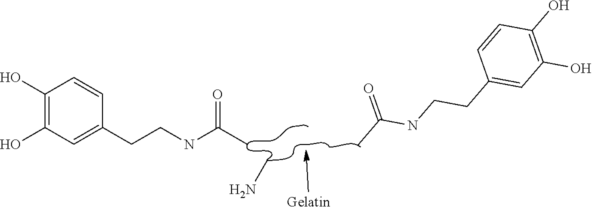

Scheme 3: schematic representation of dopamine 34 bonded via an amide bond to gelatin 26

The attachment of dopamine 34 is accomplished by the incubation of a gelatin 26 or chitosan solution 32 by addition of a crosslinker such as EDC or hexamethylene diisocyanate. The modified gelatin 26 or the modified chitosan 32 is subsequently subjected to dialysis to remove unreacted substances.

Mineralization of the organic-polymeric matrix 18:

The matrix 18 is mineralized by incubation of the coated substrates in a solution containing calcium ions (e.g., CaCl₂) for around 15 minutes at room temperature. The pH is adjusted to 9. Subsequently a phosphate-containing solution (e.g., of Na₂HPO₄) is added dropwise at a controlled rate of around 3 mL/min. It is necessary here for the pH to be kept constant at 9. When addition has been made, the solution is stirred at room temperature for a further 24 h. The substrates are subsequently washed with water.

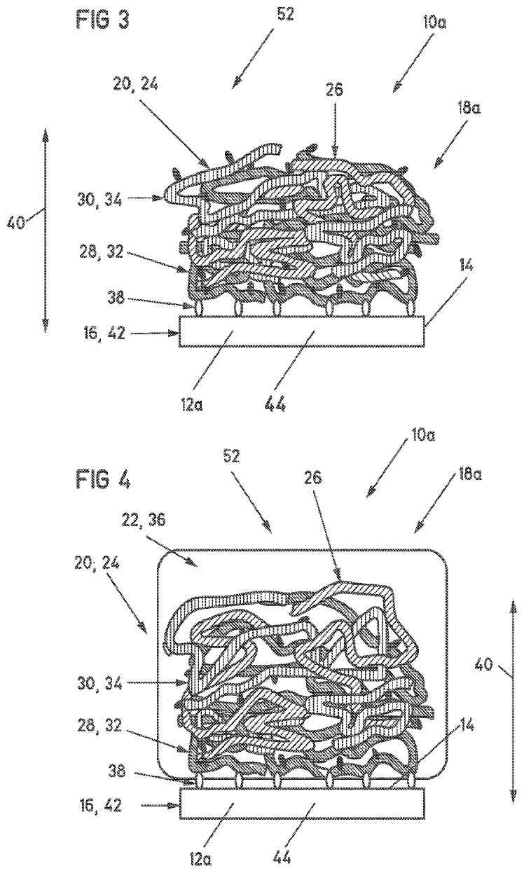

FIGS. 3 and 4 show an alternative exemplary embodiment of the organic-polymeric matrix 18. Essentially, substances, features and functions which remain the same are labeled in principle with the same reference numerals. In order to distinguish the exemplary embodiments, however, the letter a has been added to the reference numerals of the alternative exemplary embodiment. The description below is confined essentially to the differences relative to the exemplary embodiment in FIGS. 1 and 2; regarding substances, features and functions which remain the same, reference may be made to the description of the exemplary embodiment in FIGS. 1 and 2.

FIGS. 3 and 4 show a material 10a for a bone implant 12a, with an alternatively constructed organic-polymeric matrix 18a. In this case the embodiments of the examples of FIGS. 1/2 and FIGS. 3/4 differ in that, rather than the organic-polymeric matrix 18a being constructed in layers, the organic-polymeric matrix 18a is instead constructed as a mixture 52 of the individual substances, collagen/gelatin 26, polycatechol 30/polydopamine 34, polysaccharide 28/chitosan 32.

### LIST OF REFERENCE NUMERALS

10 Material
12 Bone implant
14 Surface
16 Material
18 Matrix
20 Substance
22 Calcium phosphate
24 Pyrocatechol
26 Gelatin
28 Polysaccharide
30 Polycatechol
32 Chitosan
34 Dopamine
36 Hydroxylapatite
38 Linker
40 Layer thickness
42 Titanium
44 Body
46 Layer
48 Layer
50 Layer
52 Mixture

1-15. (canceled)

16. A material for a bone implant, the material comprising:
a surface having a substance selected from the group consisting of metal-based materials, metal alloys, oxide ceramic materials, polymer materials, composite materials, and combinations thereof;
an organic-polymeric matrix bonded covalently to said surface;
a further substance incorporated or attached to said organic-polymeric matrix for binding metal ions or nanoparticles; and
calcium phosphate incorporated into said organic-polymeric matrix.

17. The material for the bone implant according to claim 16, wherein said further substance for binding the metal ions or the nanoparticles is a pyrocatechol.

18. The material for the bone implant according to claim 16, wherein said organic-polymeric matrix bonded covalently to said surface contains at least one of a collagen, a gelatin, a polysaccharide, a modified polysaccharide or a polycatechol.

19. The material for the bone implant according to claim 18, wherein said polysaccharide is selected from the group consisting of chitosan, alginate, hyaluronic acid, alginic acid, hyaluronate, pectin, carrageenan, agarose, amylose, heparin/heparan sulfate, chondroitin sulfate/dermatan sulfate, keratan sulfate, xylans or mannans after a carboxy-functionalization, xanthan, gellan, fucogalactan, and welan gum.

20. The material for the bone implant according to claim 18, wherein:
said polycatechol has a pyrocatechol main structure; and
said pyrocatechol being selected from the group consisting of dopamine, norepinephrine and L3,4-dihydroxyphenylalanine.

21. The material for the bone implant according to claim 18, wherein said calcium phosphate is selected from the group consisting of calcium orthophosphate in all mineral forms.

22. The material for the bone implant according to claim 21, wherein said calcium phosphate is selected from the group consisting of amorphous calcium orthophosphate, dicalcium phosphate dihydrate (DCPD; brushite), octacalcium phosphate, and hydroxylapatite, including with partial fluoride, chloride, strontium or carbonate substitution, and combinations thereof.

23. The material for the bone implant according to claim 16, further comprising a linker selected from the group consisting of pyrocatechol, phosphonic acid, phosphoric acid, and organosilane molecules, wherein said organic-polymeric matrix is bonded to said surface via said linker.

24. The material for the bone implant according to claim 16, wherein said organic-polymeric matrix has a layer thickness of between 0.5 micrometers and 50 μm.

25. The material for the bone implant according to claim 16, wherein said organic-polymeric matrix covers said surface entirely with said substance.

26. The material for the bone implant according to claim 16, wherein:
said substance covering said surface is titanium;
said organic-polymeric matrix bonded covalently to said surface contains pyrocatechol-modified gelatin, pyrocatechol-modified chitosan, and polydopamine;
said further substance includes pyrocatechol molecules incorporated or attached to said organic-polymeric matrix and binds the metal ions or the nanoparticles; and
said organic-polymeric matrix contains or incorporates hydroxylapatite.

27. The material for the bone implant according to claim 17, wherein said pyrocatechol is selected from the group consisting of protocatechuic alcohol, protocatechualdehyde, protocatechuic acid, 3-(3,4-dihydroxy¬phen¬yl)propionic acid, and 3,4-dihydroxyphenylacetic acid.

28. The material for the bone implant according to claim 24, wherein said organic-polymeric matrix has a layer thickness of between 1 μm and 20 μm.

29. The material for the bone implant according to claim 24, wherein said organic-polymeric matrix has a layer thickness of 10 μm.

30. A method for producing a material for a bone implant, which comprises the steps of:
a) providing a surface with a substance selected from the group consisting of metal-based materials, metal alloys, oxide ceramic materials, polymer materials, composite materials, and combinations thereof;
b) covalently coupling an organic-polymeric matrix to the surface;
c) introducing and/or coupling a further substance which binds metal ions or nanoparticles into/to the organic-polymeric matrix; and
d) mineralizing the organic-polymeric matrix with calcium phosphate.

31. The method according to claim 30, which further comprises forming the organic-polymeric matrix to contain a polycatechol, the polycatechol being prepared in step by means of oxidation of at least one pyrocatechol.

32. A bone implant, comprising:
a solid material and/or a solid body having the material for the bone implant according to claim 16 being applied thereto.

33. A method of using a material, which comprises the steps of:
providing the material according to claim 16; and
using the material as a bone implant material on a bone implant.
